(19) United States
(12) Patent Application Publication
(10) Pub. No.: US 2022/0213546 A1
(43) Pub. Date: Jul. 7, 2022

(54) PROTOCOL FOR DETECTING INTERACTIONS WITHIN ONE OR MORE DNA MOLECULES WITHIN A CELL

(71) Applicant: Oxford Nanopore Technologies Plc, Oxford (GB)

(72) Inventors: David Dai, New York, NY (US); Matthew Pendleton, New York, NY (US)

(73) Assignee: Oxford Nanopore Technologies Plc, Oxford (GB)

(21) Appl. No.: 17/612,610

(22) PCT Filed: May 22, 2020
(86) PCT No.: PCT/GB2020/051253

§ 371 Date: Nov. 19, 2021

Related U.S. Application Data
(60) Provisional application No. 62/851,543, filed on May 22, 2019.

(51) Int. Cl. C12Q 1/6876; C12Q 1/6806; C12Q 1/6855

(52) U.S. Cl. CPC C12Q 1/6876; C12Q 1/6855; C12Q 1/6806

(57) ABSTRACT

A method for detecting interactions between elements within one or more DNA molecules within a cell, wherein the elements are not adjacent in the primary DNA sequence, the method comprising: a) providing a cell in which elements within one or more DNA molecules that are in close proximity are cross-linked; b) simultaneously lysing the cell and mechanically fragmenting the DNA molecules within the cell; c) proximity ligating the one or more fragmented DNA molecules; d) reversing the crosslinks in the ligated DNA molecules; e) sequencing the ligated DNA molecules; and f) analysing the sequencing data to detect interactions between elements within the one or more DNA molecules within the cell.

### FIELD OF THE INVENTION

The present invention relates generally to a method for detecting interactions between elements within one or more DNA molecules within a cell, wherein the elements are not adjacent in the primary DNA sequence.

### BACKGROUND

There is currently a need for technologies that are capable of providing information that enables an improved understanding of genomic spatial organization. Existing technologies are slow and include steps that introduce biases. These biases prohibit resolution of DNA interactions across the entirety of a genomic sequence.

### SUMMARY

The present inventors have identified novel methods for detecting and resolving interactions between elements within one or more DNA molecules within a cell. Particularly, the methods enable the detection of interacting elements that are not adjacent in the primary DNA sequence. Interaction information may provide an understanding of conformational features underpinning the hierarchical organization of the genome. Particularly, these conformational features include whole chromosome territories, large-scale active and repressed compartments, topologically associated domains, lamin associated domains, nucleolus associated domains, and individual looping interactions between one or more elements within the same or different chromosome. The methods may also provide likewise information when applied to heterogeneous metagenomics samples, in particular identifying interactions between bacterial chromosomes and their plasmids.

A key benefit of the methods is that the simultaneous steps of mechanical fragmentation of cells and mechanical fragmentation of cross-linked DNA means that there is a reduced capacity for the introduction of errors as the number of steps in the protocol is low. Single-step cell lysis and DNA fragmentation both simplifies and streamlines the methods. Furthermore, and importantly, the methods provide sequencing-based element-interaction information that is sequence independent and thus not affected by biases in restriction enzyme targeting and the like; the methods are not affected by chemical modification of DNA bases or the accessibility of DNA sequences. Particularly, similar approaches in the art utilize restriction enzymes in the fragmentation step. The method is not affected by regions of the genome that are under- or over-concentrated with restriction enzyme motifs. Instead, mechanical fragmentation of the DNA according to the present methods enables maximal mapping of sequencing data without sacrificing resolution.

Accordingly, provided herein is a method for detecting interactions between elements within one or more DNA molecules within a cell, wherein the elements are not adjacent in the primary DNA sequence, the method comprising: a) providing a cell in which elements within one or more DNA molecules that are in close proximity are cross-linked; b) simultaneously lysing the cell and mechanically fragmenting the DNA molecules within the cell; c) proximity ligating the one or more fragmented DNA molecules; d) reversing the crosslinks in the ligated DNA molecules; e) sequencing the ligated DNA molecules; and f) analysing the sequencing data to detect interactions between elements within the one or more DNA molecules within the cell.

Also provided is a method for detecting interactions between elements within one or more DNA molecules within a cell or nucleus, wherein the elements are not adjacent in the primary DNA sequence, the method comprising: a) providing a cell or nucleus in which elements within one or more DNA molecules that are in close proximity are cross-linked; b) mechanically fragmenting the DNA molecules within the cell by bead beating; c) proximity ligating the one or more fragmented DNA molecules; d) reversing the crosslinks in the ligated DNA molecules; e) sequencing the ligated DNA molecules; and f) analysing the sequencing data to detect interactions between elements within the one or more DNA molecules within the cell.

### DESCRIPTION OF THE FIGURES

It is to be understood that Figures are for the purpose of illustrating particular embodiments of the invention only, and are not intended to be limiting.

FIG. 1 shows an example of how methods of the disclosure may be used to provide information on interaction between elements within one or more DNA molecules. The example shows a heterogeneous mixture of intact cells in a sample tube. A crosslinking agent is then applied to the intact cells to crosslink molecules inside the cells. Particularly, the crosslinking agent may crosslink DNA molecules to interacting DNA molecules, DNA molecules to interacting proteins and/or proteins to interacting proteins. The circular dotted lines represent cells, nuclei or any other vesicle. The lines labelled with ‘Genome A’, ‘Plasmid A’ and ‘Genome B’ represent DNA molecules. The small, overlapping, greyed circles represent proteins that are interacting with one another and are simultaneously interacting with the DNA molecules. The proteins in this schematic are therefore ‘bridging’ the interacting elements within one or more DNA molecules. These protein-protein and protein-DNA interactions undergo crosslinking as a result of the application of the crosslinking agent. The cells and DNA molecules are the fragmented by the mechanical process of ‘bead-beating’. Fragmented ends of the crosslinked DNA molecules are then ligated to fragmented ends that are in proximity to one another. In the top panel of the exemplary figure, the fragmented Genome A DNA molecule is ligated to the fragmented Plasmid A DNA molecule in instances whereby elements within the respective molecules, thus meaning that the fragmented ends of the respective crosslinked DNA molecules that are in close proximity to one another are ligated. The example shows that the crosslinks may then be reversed and the ligated DNA molecule may be purified. In the example, the purified ligated DNA molecule from the top panel represents a concatenated sequence of Genome A and Plasmid A sequences indicating that the elements within these sequences were interacting with one another in the original cell from which they were derived. The example further shows that purified DNA molecules may then be size selected and amplified by polymerase chain reaction (PCR), then subjected to a sequencing library preparation protocol (e.g. with the incorporation of one or more adaptors, leader sequences and/or hairpin loops) and sequencing.

FIG. 2 shows an exemplary bioinformatics analysis workflow whereby sequence reads are obtained by the method depicted by the example in FIG. 1, wherein the sequencing step to derive the sequencing reads is performed by a nanopore-based method. In FIG. 2, these sequencing reads are termed ‘Nanopore MetaPore-C reads’. Sequencing reads, as exemplified by MetaPore-C reads (concatenated sequences) are subjected to local alignment to reference genome sequences. As can be observed in the bottom left panel of FIG. 2, regions of an individual sequencing read may align to the same sequence that is present in more than one species/genome. Alignment paths through each individual MetaPore-C sequence read are therefore optimized to resolve the most likely species that the sequencing read aligns to. The example further shows that genome sequences may be segregated into ‘bins’ of a suitable length (in bp) and the aligned MetaPore-C sequencing reads may be assigned to said bins. The number of assigned reads to bins may then be used to tabulate a contact map (heat map) on the basis of the frequency by which the assigned reads neighbor one another in the MetaPore-C sequencing reads.

FIG. 3 shows data derived from exemplary methods that show the identification of intra- and extra-chromosomal contacts (interactions) in a probiotic sample. A shows a table of 15 known bacterial strains contained within an initial probiotic sample that was subjected to a method depicted in FIG. 1 for determining interactions between elements within one or more DNA molecules within a cell. B and C show contact maps representing each of the 15 bacterial strains of A prepared in accordance with the bioinformatics workflow set out in FIG. 2. D shows an average nucleotide density heat map for indicating the degree genomic similarity between the 15 bacterial strains (and their associated plasmids) of A. E and F show bar charts indicating the number of contacts and the types of contacts for each bacterial DNA molecule.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “a polynucleotide” includes two or more polynucleotides, reference to a “molecule” refers to two or more and the like.

All publications, patents and patent applications cited herein, whether supra or infra, are hereby incorporated by reference in their entirety.

### Methods

Provided is a method for detecting interactions between elements within one or more DNA molecules within a cell, wherein the elements are not adjacent in the primary DNA sequence, the method comprising: a) providing a cell in which elements within one or more DNA molecules that are in close proximity are cross-linked; b) simultaneously lysing the cell and mechanically fragmenting the DNA molecules within the cell; c) proximity ligating the one or more fragmented DNA molecules; d) reversing the crosslinks in the ligated DNA molecules; e) sequencing the ligated DNA molecules; and f) analysing the sequencing data to detect interactions between elements within the one or more DNA molecules within the cell.

Also provided is a method for detecting interactions between elements within one or more DNA molecules within a cell or nucleus, wherein the elements are not adjacent in the primary DNA sequence, the method comprising: a) providing a cell or nucleus in which elements within one or more DNA molecules that are in close proximity are cross-linked; b) mechanically fragmenting the DNA molecules within the cell by bead beating; c) proximity ligating the one or more fragmented DNA molecules; d) reversing the crosslinks in the ligated DNA molecules; e) sequencing the ligated DNA molecules; and f) analysing the sequencing data to detect interactions between elements within the one or more DNA molecules within the cell.

The methods may be used, for example, to obtain information relating to the spatial organization one or more DNA molecules (e.g. a genome) in a cell. In particular, the methods can provide information relating to the hierarchical organization of the genome in a cell. Exemplary conformational features underpinning the hierarchical organization of the genome which may be resolved by the present methods include whole chromosome territories, large-scale active and repressed compartments, topologically associated domains, lamin associated domains, nucleolus associated domains, and individual looping interactions between one or more elements within the same or different chromosome. The present methods may also provide likewise information when applied to heterogeneous metagenomics samples, in particular identifying interactions between bacterial chromosomes and their plasmids.

### Interactions Between Elements

In any of the methods described herein, “elements” may refer to a portion of nucleotide sequence of any size within one or more nucleic acid molecules. The nucleic acid molecule may be deoxyribonucleic acid (DNA) or ribonucleic acid (RNA). The nucleic acid molecule can comprise one strand of RNA hybridised to one strand of DNA.

The nucleic acid molecule is preferably DNA, RNA or a DNA or RNA hybrid, most preferably DNA. The nucleic acid molecule may be double stranded. The nucleic acid molecule may be genomic DNA. The nucleic acid molecule may comprise single stranded regions and regions with other structures, such as hairpin loops, triplexes and/or quadruplexes. The DNA/RNA hybrid may comprise DNA and RNA on the same strand. Preferably, the DNA/RNA hybrid comprises one DNA strand hybridized to a RNA strand.

The nucleic acid molecule can be any length. For example, the nucleic acid molecule can be at least 10, at least 50, at least 100, at least 150, at least 200, at least 250, at least 300, at least 400 or at least 500 nucleotides or nucleotide pairs in length. The target nucleic acid molecule can be 1000 or more nucleotides or nucleotide pairs, 5000 or more nucleotides or nucleotide pairs in length or 100000 or more nucleotides or nucleotide pairs in length. The nucleic acid molecule can be an entire genome. The nucleic acid molecule can be the entirety of all nucleic acid molecules comprised within a cell. The nucleic acid molecule can be a sub-selection of all of the nucleic acid molecules comprised within a cell. The nucleic acid molecule can be the entirety of the DNA comprised within a cell. The nucleic acid molecule can be a sub-selection of the DNA comprised within a cell, for example an individual chromosome.

Elements may be a portion of nucleotide sequence of any size within one or more nucleic acid molecules. An element may be a locus defined by specific coordinates in accordance with a given genome reference assembly. Elements may therefore be loci within one or more chromosomes. An element may be a coding or non-coding sequence of a genome. An element may be a nucleotide sequence within heterochromatin (closed chromatin) or euchromatin (open chromatin). An element may be a cis-regulatory element or cis-regulatory module. An element may be a promoter, an enhancer, a silencer, an exon, an intron. An element may be binding site for a protein, for example a histone protein, a transcription factor and/or a trans-acting factor. An element may be a portion of open chromatin flanked by histones. An element may be a CpG island. An element may be a gene desert. An element may be a transcription factor binding motif. An element may be region comprising small nucleotide polymorphisms in linkage disequilibrium with one another. An element may be a single SNP, a CpG, or a single nucleotide base. In any of the methods described herein, the elements are not adjacent in the primary nucleotide sequence of the one or more DNA molecules.

In any of the methods described herein, “interactions” may refer to any form of direct or indirect contact between elements within one or more DNA molecules. The elements may be comprised within one or more DNA molecules within one or more cells. The interactions may refer to indirect or direct interactions between elements, wherein the elements are not adjacent in the primary DNA sequence. The interactions may therefore provide an indication of 3D genome architecture. In particular, the interaction may further provide an indication of a precise map of the special organization of elements within a DNA molecule. An interaction may be two or more elements in proximity to one another. Thus, in the context of describing the spatial organization of the elements within a DNA molecule, elements that are proximal to one another can be considered to also be interacting, regardless of whether there is any functional consequence to the proximity of the two or more elements. The term “proximity” when used in the present context refers to the distance in three-dimensional space between the two elements. For example, DNA sequence elements in a chromosome that are close (e.g. within about 10, 50, 100, 150, 200, or 250 bp or more) in primary sequence are always in close proximity to each other. In some instances, DNA sequence elements that are distant in primary sequence in a chromosome (e.g., separated by more than about 200; 250; 300; 400; 500; 1000; 1500; 2000; 5000; 10,000; 25,000; 50,000; 100,000; 250,000; 500,000; or 1,000,000 bp) can be in close proximity to each other due to the tertiary or quaternary structure of the chromosome(s). In some instances, DNA sequence elements that lie on different chromosomes can be in close proximity to each other due to the quaternary structure of the chromosomes. In some instances, nucleic acid sequence elements are distal with respect to primary sequence because one or more elements are chromosomal DNA sequence elements and one or more other elements are RNA (or cDNA) sequence elements. As such, the nucleic acid sequence elements can be, or can be within, different nucleic acid molecules. In such cases, the two or more nucleic acid sequence elements can be in close proximity to each other due to their formation of a complex. For example, non-coding RNAs can associate with one or more DNA sequence elements in a genome. Thus, in any of the methods described herein, DNA sequence elements may be considered to be interacting as a consequence of their proximity.

Two or more elements may be interacting simultaneously. Elements may be directly interacting or may be interacting indirectly. Indirect interactions may be mediated by direct interactions with one or more proteins. For example, an indirect interaction may be represented by a protein complex that is simultaneously bound to an enhancer and to a promoter, wherein the two elements are more than 100,000 bp away from one another in terms of primary sequence.

### Sample

In any of the methods described herein, the sample may be any suitable sample. The sample should contain one or more DNA molecules. The sample is typically one that is known to contain or is suspected of containing one or more DNA molecules. The sample may contain one or more cells.

The sample may be a biological sample. The disclosed methods may be carried out in vitro on a sample comprising cells from any organism or microorganism. The organism or microorganism is typically archaean, prokaryotic or eukaryotic and typically belongs to one of the five kingdoms: plantae, animalia, fungi, monera and protista. The methods may be carried out in vitro on a sample obtained from or extracted from any virus.

The sample is preferably fluid-based. The sample typically comprises a body fluid. The body fluid may be obtained from a human or animal. The human or animal may have, be suspected of having or be at risk of a disease. The sample may be urine, lymph, saliva, mucus, seminal fluid or amniotic fluid, but is preferably whole blood, plasma or serum. Typically, the sample is human in origin, but alternatively it may be from another mammal such as from commercially farmed animals such as horses, cattle, sheep or pigs or may alternatively be pets such as cats or dogs.

Alternatively a sample of plant origin is typically obtained from a commercial crop, such as a cereal, legume, fruit or vegetable, for example wheat, barley, oats, canola, maize, soya, rice, bananas, apples, tomatoes, potatoes, grapes, tobacco, beans, lentils, sugar cane, cocoa, cotton, tea or coffee.

The sample may be a non-biological sample. The non-biological sample is preferably a fluid sample. Examples of non-biological samples include surgical fluids, water such as drinking water, sea water or river water, and reagents for laboratory tests. For example, the sample may be an environmental sample comprising a heterogeneous mixture of cells from two or more different organisms.

The sample may be processed prior to being applied to the methods described herein, for example by centrifugation or by passage through a membrane that filters out unwanted molecules or cells, such as red blood cells. The sample may be measured immediately upon being taken. The sample may also be typically stored prior to assay, preferably below −70° C.

The sample preferably comprises genomic DNA. The sample may be one or more nuclei.

### Cross-Linking

In any of the methods described herein, a sample is provided in which elements within one or more DNA molecules that are in close proximity are cross-linked. The sample may comprise one or more cells. Cells may be cross-linked by any cross-linking agent that is suitable for application to the methods described herein. A ‘snapshot’ of the spatial organization of DNA molecules, and thus the interactions between elements within one or more DNA molecules within a cell, can be obtained by applying a cross-linking agent to the cell. One or more crosslinking agents can be applied to the cells within the sample to covalently bond molecules that are in proximity to one another. Preferably, the one or more cross-linking agents will cross-link DNA to DNA, DNA to proteins, and proteins to proteins. Further preferably, the methods may further comprise cross-linking the DNA molecules and/or DNA-interacting proteins.

Preferably, cross-linking agents in the methods described herein react with amino groups in proteins, and/or imino and amino groups in DNA, thus being capable of forming crosslinks between any one or all of these groups. Exemplary cross-linking agents include formaldehyde, disuccinimidyl glutarate (DSG), Bis[2-(N-succinimidyl-oxycarbonyloxy)ethyl] sulfone (BSOCOES), Disuccinimidyl Dibutyric Urea (DSBU), 1,5-difluoro-2,4-dinitrobenzene (DFDNB), Dimethyl adipimidate dihydrochloride (DMA), dimethyl pimelimidate (DMP), dimethyl suberimidate (DMS), dithiobis(succinimidyl propionate) (DSP), disuccinimidyl suberate (DSS), disuccinimidyl sulfoxide (DSSO), disuccinimidyl tartrate (DST), Dimethyl dithiobispropionimidate (DTBP), ethylene glycol bis(succinimidyl succinate) (EGS), sulfo-EGS, tris-(succinimidyl)aminotriacetate (TSAT). Preferably, the cross-linking is achieved by treating the one or more cells with a cross-linking agent. Even more preferably, the cross-linking is achieved by treating the cells with formaldehyde and/or disuccinimidyl glutarate (DSG).

The conditions of the cross-linking step may be appropriately chosen by the skilled person. For example, it is within the routine skill of a person skilled in the art to select an appropriate buffer and/or temperature to achieve a desired degree of cross-linking using any given cross-linking agent.

### Lysing and Fragmenting

In any of the methods described herein, the cross-linked cells may be lysed. DNA molecules from the cells may be fragmented at the same time as or after cell lysis. In any of the methods described herein, the cross-linked cells may be lysed whilst, simultaneously, the DNA molecules within the cell are fragmented.

The cells may be lysed by any suitable protocol. Cell lysis may be performed by a physical disruption or solution-based protocol. The physical disruption protocol may be a ‘mechanical’ protocol. Physical disruption and mechanical protocols may fragment lyse cells whilst simultaneously fragmenting DNA molecules comprised within the cells. An exemplary physical disruption protocol applicable to the disclosed methods is the use of a waring blender polytron. Another exemplary physical disruption protocol applicable to the disclosed methods is the use of a Dounce homogenizer. Another exemplary physical disruption protocol applicable to the disclosed methods is the use of a Potter-Elevehjem homogenizer. Another exemplary physical disruption protocol applicable to the disclosed methods is the use of sonication. Another exemplary physical disruption protocol applicable to the disclosed methods is the use of freeze-thaw cycling. Another exemplary physical disruption protocol applicable to the disclosed methods is the use of a pestle and mortar. A preferable example of physical disruption applicable to the disclosed methods is bead-beating.

Bead-beating comprises combining beads with a sample and physically agitating the combination, thus leading to fragmentation of the cells in the sample. The beads used in bead beating can be of any suitable material for application to the methods described herein. For example, beads may be ceramic, metal or glass. Preferably, the beads are glass. The beads may be any size suitable for applying to a sample in a mechanical physical disruption protocol. The beads may be less than 5 mm diameter. The beads may be less than 3 mm diameter. The beads may be less than 1 mm diameter. Preferably the beads are between 0.1 mm and 1 mm diameter. Even more preferably, the beads are 0.5 mm diameter. The combined sample and beads may be agitated by any suitable method depending on the volume of the sample in addition to the size and amount of beads used. Agitation of cells by bead-beating may lyse cells and fragment DNA molecules comprised within the cells simultaneously.

An exemplary method of agitation is by vortexing. Any standard laboratory benchtop vortex may be used. Preferably the vortex has intensity settings. Preferably, the agitation step is performed at the highest intensity selectable on the standard laboratory benchtop vortex. Any bead-beating step in the methods described herein may be performed by one single period of agitation. The period of agitation may be less than 30 minutes. The period of agitation may be less than 20 minutes. The period of agitation may be 15 minutes.

Any bead-beating step in the methods described herein may be performed by cycles of agitation and cooling. For example, the bead-beating step may involve 5 cycles of agitation separated by cooling incubation steps. The cycles of agitation may be for any suitable period of time. The separation cooling incubation steps may be for any suitable period of time.

The sample that is being subjected to bead-beating is preferably kept cool during the bead-beating protocol. The bead-beating may be performed at below room temperature. The bead-beating may be performed at below 18° C. The bead-beating may be performed at 4° C. The sample being subjected to bead-beating may be incubated at a temperature below room temperature between agitation steps. The sample being subjected to bead-beating may be incubated at a temperature below 18° C. between agitation steps. The sample being subjected to bead-beating may be incubated at 4° C. between agitation steps. Preferably, the bead-beating comprises three 3-minute vortexing steps, wherein the vortexing is performed at the highest intensity selectable on the standard laboratory benchtop vortex, each separated by 2-minute incubations at 4° C. or on ice.

DNA molecules that were comprised within the cells that were subjected to lysis may then be fragmented. Although, in any of the methods described herein, the cross-linked cells may be lysed whilst, simultaneously, the DNA molecules within the cells are fragmented. Simultaneous steps of mechanical fragmentation of cells and mechanical fragmentation of cross-linked DNA means that there is a reduced capacity for the introduction of errors as the number of steps in the protocol is low. Single-step cell lysis and DNA fragmentation both simplifies and streamlines the methods.

The described bead-beating parameters may, for example, be combined in any way to achieve cell lysis and/or the desired degree of fragmentation of DNA.

DNA molecules may be fragmented by any suitable method. DNA fragmentation comprises breaking DNA molecules into smaller pieces. The DNA molecules may be derived from cells or nuclei that have been lysed. Preferably, cells are lysed and DNA molecules comprised within the cells are fragmented simultaneously in a single step.

DNA may be fragmented mechanically. Preferably, the method comprises lysing cells in which elements within one or more DNA molecules that are in close proximity are crosslinked and simultaneously mechanically fragmenting the DNA molecules within the cells. Preferably, the cells are mechanically lysed and the DNA molecules are mechanically fragmented. Even more preferably, cells are lysed mechanically and DNA molecules comprised within the cells are fragmented mechanically simultaneously in a single step. Any of the above described mechanical methods of lysing cells may fragment the DNA.

The methods of the disclosure may comprise mechanical fragmentation by bead-beating. Bead beating may be applied to, for example, intact cells, intact nuclei, lysed cells, lysed nuclei and/or isolated DNA. Preferably, the cross-linked cells of the methods described herein are lysed whilst, simultaneously, the DNA molecules within the cells are fragmented. A longer duration of bead-beating, or a greater intensity of bead-beating would lead to the DNA molecules being fragmented into smaller pieces. The simultaneous steps of mechanical fragmentation of cells and mechanical fragmentation of cross-linked DNA means that there is a reduced capacity for the introduction of errors as the number of steps in the protocol is low.

Single-step cell mechanical lysis and mechanical DNA fragmentation both simplifies and streamlines the methods. Furthermore, the disclosed methods provide DNA fragments that have been obtained in a sequence-independent manner. This means that the fragmentation steps of the method involving mechanical fragmentation such as bead-beating will not be affected by over- or under-represented restriction enzyme motifs or chemical modifications of DNA. Thus, the lack of biases in the mechanical fragmentation steps of the present methods enables improved genomic coverage when detecting interactions between elements. Mechanical fragmentation of the DNA according to the present methods enables maximal mapping of sequencing data without sacrificing resolution. By varying aspects of a mechanical fragmentation step (e.g. bead-beating), fragment size can be fine-tuned.

The DNA molecules may be fragmented to any size suitable for the chosen sequencing platform to be applied to the methods described herein. The mechanical fragmentation step may generate DNA molecules that are at least about 100 bp, for example at least about 250 bp, at least about 500 bp, at least about 1 kbp, at least about 2 kbp, at least about 5 kbp, at least about 10 kbp, or at least about 15 kbp. For example the fragments may have lengths of from about 100 bp to about 15 kbp, such as, for example, from about 250 bp, about 500 bp, about 1 kbp or about 2 kbp up to about 5 kbp, about 10 kbp or about 15 kbp.

### Proximity Ligation

In any of the methods described herein, fragmented DNA molecules may be proximity ligated. Proximity ligation in the present context has the effect of forming concatemer sequences whereby DNA fragments representative of elements within the original one or more DNA molecules become covalently ligated to other fragments that are in proximity in three-dimensional space but not adjacent in primary sequence. Concatemer sequences thus indicate what DNA elements are interacting with one another within one or more DNA molecules.

Following the step of fragmentation, the fragmented DNA molecule sample is preferably diluted. The absence of dilution could lead to spurious proximity ligation events with cross-linked fragmented DNA molecules that are randomly in proximity other cross-linked fragmented DNA molecules in solution. Alternative to diluting the sample solution, DNA fragments may be separated on an agarose gel on the basis of their size, thus reducing the likelihood of spurious proximity ligation events with cross-linked fragmented DNA molecules that are randomly in proximity other cross-linked fragmented DNA molecules.

The proximity ligation step in any of the methods described herein can be performed with any suitable DNA ligase known in the art. Exemplary ligases and kits may include one or more of T4 DNA ligase, Tfi DNA ligase, DNA ligase I, DNA ligase II, DNA ligase III, DNA ligase IV, a small footprint DNA ligase, NEB's Blunt/TA master mix or NEB's Quick Ligation™ Kit. Preferably, the ligase is capable of ligating blunt ends of double stranded DNA fragments.

In any of the methods described herein, proximity ligation is performed to provide a 5C or Hi-C library.

Prior to the step of proximity ligation, but following the step of DNA molecule fragmentation, the still cross-linked DNA fragmented DNA molecules may undergo ‘end-repair’. DNA fragment end repair may facilitate proximity ligation. Any suitable DNA end-repair protocols may be used. An exemplary product for use in repairing fragmented DNA ends is the NEBNext End Repair enzyme. Preferably, the fragmented DNA molecules are ‘blunt-ended’ to provide DNA fragments with blunt ends for proximity ligation. Any enzyme or kit capable of blunt-ending double stranded DNA molecules may be used in the present method.

In any of the methods described herein, following the step of proximity ligation, cross-links may be reversed by any suitable method. The method of cross-link reversal suitable for application to the methods described herein may depend of the one or more cross-linking agents used in the cross-linking step of the method described herein. For example, wherein formaldehyde is the cross-linking agent used in a method described herein, cross-links may be reversed either by incubation with high salt (e.g. NaCl) and prolonged incubation at 65° C., or by incubation with Tris HCl buffer combined with RNaseA and proteinase K for a prolonged period at 65° C.

The ligated DNA fragments may, for example, have lengths of at least about 250 bp, at least about 500 bp, at least about 1 kbp, at least about 2 kbp, at least about 5 kbp, at least about 10 kbp, or at least about 15 kbp. For example the fragments may have lengths of from about 250 bp to about 100 kbp, such as, for example, from about 2 kbp, about 5 kbp, or about 10 kbp up to about 15 kbp, about 50 kbp or about 100 kbp.

### DNA Purification

In any of the methods described herein, DNA molecules may be purified after the steps of proximity ligation and cross-link reversal. In any of the methods described herein, DNA can be performed by any methods known in the art that are suitable for purifying DNA. Preferably, purification methods applied to the present methods provide DNA that is sufficiently pure for sequencing. Exemplary methods for purifying DNA include organic extraction methods such as phenol-chloroform and ethanol precipitation, Chelex extraction purification, and solid phase purification, and any known DNA purification kits in the art. Preferably, purification steps to be used in the methods described herein use solid phase reversible immobilization (SPRI) beads.

### Size Selection

Any of the methods described herein may comprise a step of selecting DNA of a desired size at any suitable stage. Selecting fragments of a desired size may be performed after fragmenting the cross-linked DNA molecules, and/or after proximity ligating the one or more fragmented DNA molecules, and/or reversing the cross-links in the ligated DNA molecules. Size selection may be performed by any suitable method. In any of the methods described herein, any inclusion of a step comprising selecting DNA fragments of a desired size will preferably take place immediately prior to any sequencing step. Exemplary DNA size selection methods include separation of DNA fragments on an agarose gel followed by excision of the gel comprising the desired size of DNA fragments and purification, SPRI beads or BluePippin (Sage Science).

The desired size of DNA fragments may vary depending upon what sequencing platform is to be used to sequence the ligated DNA molecules. Fragments sizes of between 200-500 bp are preferred for Illumina-based sequencing methods. When applying a sequencing methods from Oxford Nanopore Technologies to the methods described herein, the desired size of DNA fragments are typically more than 500 bp, preferably more than 1 kb, and even more preferably more than 3 kb.

For example, Oxford Nanopore Technologies' sequencing platforms may be used to sequence DNA molecules longer than 3 kb. Preferably, the ligated fragments that form a concatemer DNA fragment are long enough to be uniquely mapped to a reference genome assembly. Even more preferably, ligated fragments that form a concatemer DNA fragment are long enough and of sufficiently high read quality to be uniquely mapped to a reference genome assembly.

### Enrichment

The methods disclosed herein may further comprise a step of enriching for one or more DNA molecules of interest. DNA molecules of interest may be enriched at any stage considered suitable in the methods. In some instances, the ligated DNA molecules may be enriched prior to sequencing. In other instances, the ligated DNA molecules may be enriched immediately prior to sequencing. In other instances, the ligated DNA molecules may be enriched after being purified. In other instances, the ligated DNA molecules may be enriched after selecting DNA fragments of a desired size. In other instances, the ligated DNA molecules may be enriched after purification and size selection.

DNA molecules of interest may be enriched by any suitable method. A DNA molecule of interest may, for example, be a specific element whose interacting partners are of interest.

An exemplary method of enrichment is by hybridizing one or more labelled oligonucleotides of complementary base sequence to one or more specific regions of interest within DNA, wherein the label is an affinity tag, and further wherein the DNA molecules of interest are isolated, and therefore enriched, by targeting the affinity tag with a binding partner of the affinity tag, and discarding any DNA that is not associated with the binding partner. An exemplary affinity tag and binding partner is biotin and streptavidin.

A further exemplary method of enrichment is by inverse polymerase chain reaction (PCR). Enrichment by inverse PCR in the context of the present method may comprise circularising the ligated DNA molecules, and further wherein a pair of primer sequences of complementary base sequence to specific target regions of the circularized DNA molecule (thus, elements of the one or more DNA molecules) prime PCR extension in reverse directions, and wherein the target region and its flanking (interacting) sequences are amplified.

A further exemplary method of enrichment is by semi-specific PCR. Enrichment by semi-specific PCR in the context of the present method may comprise treating the ligated DNA molecules with end-preparation enzyme mix to create dA-tailed, ligatable ends, wherein the ligatable ends may then be ligated to universal PCR adaptors, and further wherein a sequence-specific primer to a target element of the DNA molecule can be combined with a single universal PCR primer that is comprised within the PCR adaptors, and amplifying the target and its flanking (interacting) sequences. Either side of the target may be investigated with a corresponding primer design.

### Adaptors

Any of the methods described herein may further comprise a step of adding adaptors to the ends of the DNA fragments prior to sequencing the DNA. Preferably, the adaptors are sequencing adaptors. The sequencing adaptors may be PCR sequencing adaptors. Any suitable sequencing adaptors may be applied to the methods described herein, depending on the sequencing platform used. Any suitable sequencing platform may be used in the methods described herein. More preferably, adaptors that are compatible with Oxford Nanopore Technologies' sequencing platforms are used in the methods described herein.

An Oxford Nanopore Sequencing adaptor may comprise at least one single stranded polynucleotide or non-polynucleotide region. For example, Y-adaptors for use in nanopore sequencing are known in the art. A Y adaptor typically comprises (a) a double stranded region and (b) a single stranded region or a region that is not complementary at the other end. A Y adaptor may be described as having an overhang if it comprises a single stranded region. The presence of a non-complementary region in the Y adaptor gives the adaptor its Y shape since the two strands typically do not hybridise to each other unlike the double stranded portion. The Y adaptor may comprise one or more anchors.

The Y adaptor preferably comprises a leader sequence which preferentially threads into the pore. The leader sequence typically comprises a polymer. The polymer is preferably negatively charged. The polymer is preferably a polynucleotide, such as DNA or RNA, a modified polynucleotide (such as abasic DNA), PNA, LNA, polyethylene glycol (PEG) or a polypeptide. The leader preferably comprises a polynucleotide and more preferably comprises a single stranded polynucleotide. The single stranded leader sequence most preferably comprises a single strand of DNA, such as a poly dT section. The leader sequence preferably comprises the one or more spacers.

The leader sequence can be any length, but is typically 10 to 150 nucleotides in length, such as from 20 to 150 nucleotides in length. The length of the leader typically depends on the membrane-embedded nanopore used in the method.

The leader sequence preferentially threads into the transmembrane pore and thereby facilitates the movement of polynucleotide through the pore.

The Y adaptor may comprise a capture sequence, affinity tag or pore tether that is revealed when a double stranded region to which the adaptor is attached is unwound. The capture sequence or tag functions to prevent the second strand of a DNA molecule from diffusing away from a nanopore when the DNA molecule is unwound as the first strand of the DNA molecule passes through a pore, wherein the pore binds to the tether or is tagged with an oligonucleotide comprising a sequence that is complementary to the capture sequence in the Y adaptor, an affinity partner of the tag on the Y-adaptor. The adaptor may be ligated to the DNA molecule using any method known in the art. One or both of the adaptors may be ligated using a ligase, such as T4 DNA ligase, E. coli DNA ligase, Taq DNA ligase, Tma DNA ligase and 9° N DNA ligase. Alternatively, the adaptors may be added to the DNA molecule using the methods discussed below.

In one embodiment, the method comprises modifying the one or more DNA molecules in the sample so that they comprise the Y adaptor at one end and the hairpin loop at the other end. Any manner of modification can be used.

Hairpin loop adaptors for use in nanopore sequencing are known in the art. A hairpin loop may be provided at one end of DNA molecule, the method preferably further comprises providing the DNA molecule with a hairpin loop at one end of the DNA molecule. The two strands of the DNA molecule may be joined at one end with the hairpin loop.

### Sequencing

The methods described herein may further comprise a step of sequencing the ligated DNA molecules. The step of sequencing the ligated DNA molecules may be for the purposes of determining its entire, or a portion of, its sequence. Any suitable sequencing techniques may be employed to determine the sequence of the ligated DNA molecules. In the methods of the present disclosure, the use of high-throughput, so-called “second generation”, “third generation” and “next generation” techniques may be used to sequence the ligated DNA molecules.

In second generation techniques, large numbers of DNA molecules are sequenced in parallel. Typically, tens of thousands of molecules are anchored to a given location at high density and sequences are determined in a process dependent upon DNA synthesis. Reactions generally consist of successive reagent delivery and washing steps, e.g. to allow the incorporation of reversible labelled terminator bases, and scanning steps to determine the order of base incorporation. Array-based systems of this type are available commercially e.g. from Illumina, Inc. (San Diego, Calif.).

Third generation techniques are typically defined by the absence of a requirement to halt the sequencing process between detection steps. For example, the base-specific release of hydrogen ions, which occurs during the incorporation process, can be detected in the context of microwell systems (e.g. the Ion Torrent system available from Life Technologies). Similarly, in pyrosequencing the base-specific release of pyrophosphate (PPi) is detected and analysed. In nanopore sequencing technologies, DNA molecules are passed through or positioned next to nanopores, and the identities of individual bases are determined following movement of the DNA molecule relative to the nanopore. Systems of this type are available commercially e.g. from Oxford Nanopore Technologies. In another technique, a DNA polymerase enzyme is confined in a “zero-mode waveguide” and the identity of incorporated bases determined with fluorescence detection of gamma-labeled phosphonucleotides (see e.g. Pacific Biosciences).

The methods described herein may comprise analyzing sequencing data to detect interactions between elements within the one or more DNA molecules within the cells. Analysing the sequencing data may comprise identifying concatenated sequences from different elements within the one or more DNA molecules thereby detecting interacting elements of one or more DNA elements.

The following non-limiting Examples illustrate the invention and are not intended to be limiting.

### Example 1

This Example describes an exemplary laboratory workflow applicable to the present disclosure, a method for determining interactions between elements within a cell. In particular, methods are used to investigate interactions between genomic elements that are not adjacent in the primary sequence. In addition to simplifying assembly, when applied to metagenomic samples, the disclosed methods also provide a way to associate plasmids with their host genomes. Alternative protocols for determining interactions between elements within one or more DNA molecules use restriction digestion to fragment cross-linked DNA prior to performing proximity ligation. However, restriction digestion is time-consuming and also, the choice of restriction enzyme is influenced by the nucleotide composition of the genomes in the sample, which is not always known in advance—particularly when performing metagenomics investigations. The present disclosure provides a method that avoids restriction digestion by using mechanical fragmentation, for example bead-beating, to simultaneously lyse cells and fragment DNA. Bead beating may also be used to fragment DNA from lysed cells.

### Materials and Methods

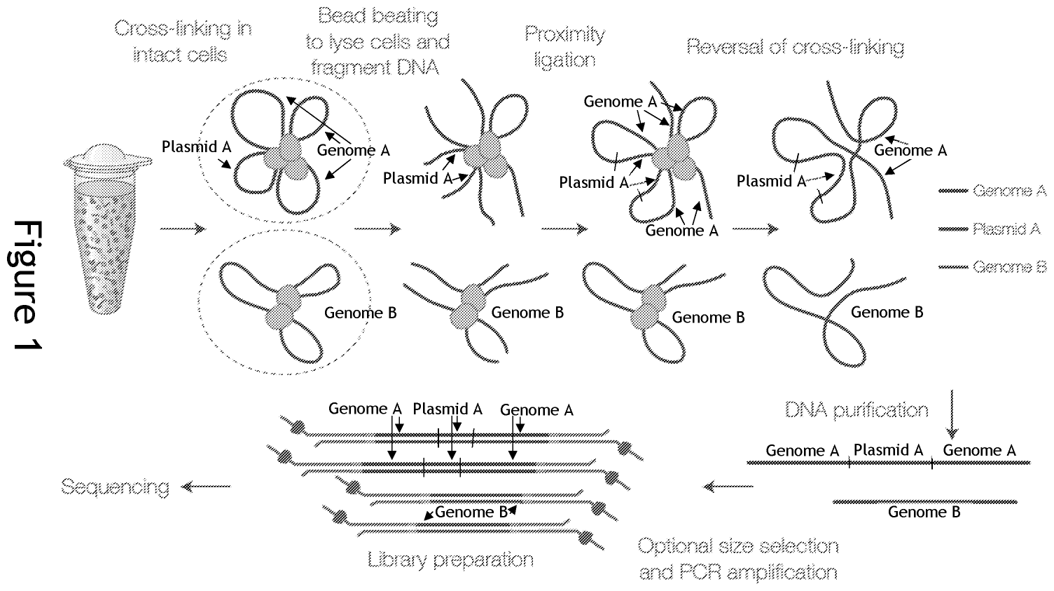

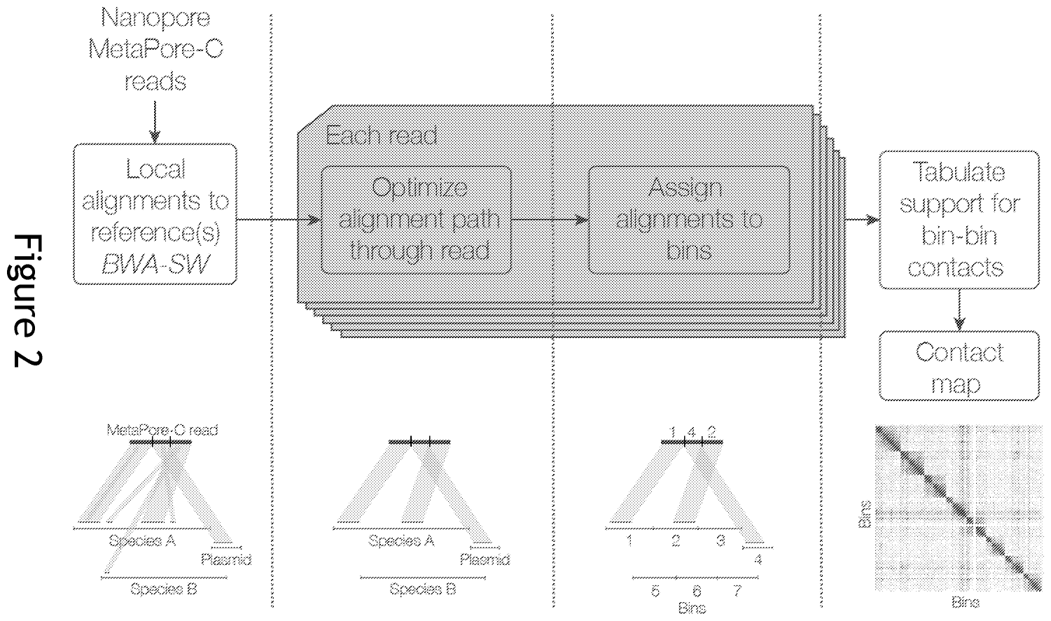

A representative schematic of the presently described exemplary method is provided in FIG. 1. In more detail, 10⁹ intact microbial cells were collected and pelleted by centrifugation (15000 g for 5 minutes). Cells were then washed once with PBS, and pelleted again by the same centrifugation procedure. Cells were re-suspended in a pre-mixed buffer: 1.2 mL PBS+34 uL 37% formaldehyde (1% final formaldehyde concentration) and allowed to incubate at room temperature for 30 minutes to crosslink DNA and proteins. 170 ul 1M glycine solution (125 mM final concentration) was added and the mixture was allowed to incubate for a further 30 minutes to so that the glycine quenched the crosslinking reaction. Cells were then pelleted by centrifugation (15000 g for 5 minutes) and the supernatant was discarded. The pelleted cells could then be stored at −20° C. for future use.

The cell pellet (˜10 μl) was then re-suspended in 200 μl bead beating solution (200 μl 1×TBS, 2 μl 100× Halt Protease Inhibitor (Thermofisher), 2 μl Triton X-100). 100 μl 0.5 mm diameter glass beads (Qiagen) were then added to the suspension and the suspension was then vortexed (VWR® Vortexer Mini 120v) for 3×5 min at highest speed, each separated by 2 minutes of incubation on ice. The step of bead beating creates free DNA ends for the following proximity ligation step. In contrast, most HiC preps use restriction enzymes digestion for the same purpose, which is subject to genome coverage biases, lower resolution and more complex laboratory steps. The suspension was then briefly spun down to separate the glass beads. The cell lysate at this point is found within the supernatant. The lysate was then transferred to a new tub and further centrifuged (15000 g for 5 minutes), following which, the supernatant was discarded. The pellet was then re-suspended in 500 μl 1×TBS and further centrifuged (17000 g for 5 minutes). The supernatant was discarded and the pellet was re-suspended in 200 μl H₂O. The re-suspended fragmented DNA was then diluted 10× and its concentration then measured using a Qubit (Thermofisher). 1 to 5 μg of the re-suspended fragmented DNA was then subjected to DNA end-repair (‘blunt-ending’) the DNA fragments produced by the earlier step of bead beating. The reaction mixture for blunt-ending was as described in Table 1. The reaction was incubated at 20° C. for 30 minutes.

TABLE 1

| 1 to 5 μg of fragmented DNA | x | μL |
|---|---|---|
| NEBNext End Repair buffer 10× | 10 | μL |
| Enzyme mix | 5 | μL |
| H₂O | 85-x | uL |
| Total | 100 | μL |

Following the 30 minutes incubation, the reaction mixture was centrifuged at max speed on a table-top centrifuge for 5 minutes, and the supernatant was subsequently discarded. The pellet was re-suspended in 200 μl of water. The re-suspended end-repaired DNA was then diluted 10× and its concentration then measured using a Qubit.

The end-repaired DNA was then subjected to proximity ligation. T4 ligase ligation reactions were set up with a DNA concentration of 1-2 ng/μl. The reaction mixture for the T4 ligase ligation reaction was as described in Table 2. The reaction was incubated at room temperature for 4 hours with occasional mixing.

TABLE 2

| | | |
|---|---|---|
| 10 × T4 ligase buffer | 100 | μL |
| DNA | 1-2 | μg |
| Water | To 1000 | μL |
| T4 DNA ligase | 10 | μL |
| (NEB, 2M units/mL) | | |

Following the 4 hours incubation time, the reaction mixture was centrifuged at 17000 g for 5 minutes. 750 μl of supernatant was then removed without disturbing the pellet. 40 μl of 5M NaCl was then added, and the mixture was vortexed to resuspend the pellet. The mixture was incubated at 65° C. overnight to deactivate the T4 ligase and decrosslink. Concatemer DNA molecules should have formed following the proximity ligation step of the method. Concatemer products should therefore be visible for QC purposes by agarose gel electrophoresis.

Multiple 250 μl ligated DNA solutions may now combined if desired. 5 μl of Triton-x100 and 50 μl of 10% Tween-20 was then added to per 250 μl ligated DNA solution. Water was then added to a total volume of 1 ml. To clean up the DNA, 45 μl proteinase-K solution (Qiagen) and 2 μl of 100 mg/ml RNaseA solution was added to the ligated DNA solution and incubated for 30 minutes at 37° C. 350 μl of Qiagen buffer B2 was then added to the reaction whilst incubating at 50° C. for a further 30 minutes.

Phenol:chloroform extraction and ethanol precipitation was then performed in order to purify the DNA cleaned-up DNA. DNA was then assessed for quality by nanodrop and agarose gel electrophoresis, and was quantified by utilisation of a Qubit.

For amplification of the purified DNA, PCR template preparation was performed. The purified DNA was treated with FFPE (NEB) and Ultra-II end-prep module (NEB). The reaction mixture was as described in Table 3.

TABLE 3

| DNA in H₂O | x | μL |
|---|---|---|
| | (100 ng-500 ng) | |
| NEBNext FFPE DNA repair buffer | 3.5 | μL |
| NEBNext FFPE DNA repair mix | 2 | μL |
| UltraII End-prep reaction buffer | 3.5 | μL |
| UltraII End-prep enzyme mix | 3 | μL |
| Total | 60 | μL |

The reaction mixture was mixed, spun down, and incubated in a thermal cycler for 15 minutes at 20° C. followed by 5 minutes at 65° C. The reaction was cleaned-up with 0.4× SPRI beads (24 μl SPRI per 60 μl reaction, for example) to size select for DNA longer than 1 kb. PCR sequencing adaptors compatible with Oxford Nanopore Technologies' sequencing platform were then ligated to the DNA. The DNA was then cleaned-up by utilisation of 0.4× SPRI beads.

For an optimal quantity of DNA for subjecting to nanopore, the DNA sample should not have an abundance of high molecular weight amplicon. Pilot PCR experiments are therefore recommended in order to determine the optimal PCR cycle number, whereby cycles of 8× to 12× are initially performed and subsequently visualised via agarose gel electrophoresis. Multiple 25 μl PCR reactions may be performed as in Table 4.

TABLE 4

| | Volume | |
|---|---|---|
| DNA template | 5-10 | ng |
| ONT PCR primer | 0.5 | μL |
| mixture (PRM) | | |
| H₂O | to 12.5 | μL |
| NEB long amp hotstart | 12.5 | μL |
| 2 × mastermix | | |
| Total | 25 | μL |

For nanopore-based sequencing application, the optimal PCR cycle DNA products were then size selected by gel-extraction or Bluepippin (SAGE Science) for PCR products larger than between 2 and 3 kb. Nanopore-based sequencing was then performed according to nanopore library preparation and sequencing protocols known in the art.

### Bioinformatics Workflow

A representative schematic of a bioinformatics analysis workflow that is applicable to the presently described exemplary method is provided by FIG. 2. In any of the described methods, the bioinformatics analysis workflow may be utilised for obtaining a metagenomics contact map from sequencing data derived using the methods applied to a metagenomics sample. In more detail, nanopore sequencing data is generated by the earlier described method to provide nanopore sequencing reads. The reads are first aligned to a collection of reference sequences for chromosomal and extra-chromosomal sequences, such as plasmids, using BWA-SW (Li H. and Durbin R. (2010) Fast and accurate long-read alignment with Burrows-Wheeler. Transform. Bioinformatics, Epub. [PMID: 20080505]). Each aligned read is filtered to retain the minimal collection of alignments that traverse the majority of the read. The reference genomes are then divided into equally sized bins and each aligned segment of a nanopore sequencing read is assigned a bin. Finally, the total number of bin-to-bin contacts is calculated from all nanopore sequencing reads and visualised in a contact map. Extra-chromosomal elements can be assigned to their host by determining which chromosome(s) share the most contacts with the element.

### Results

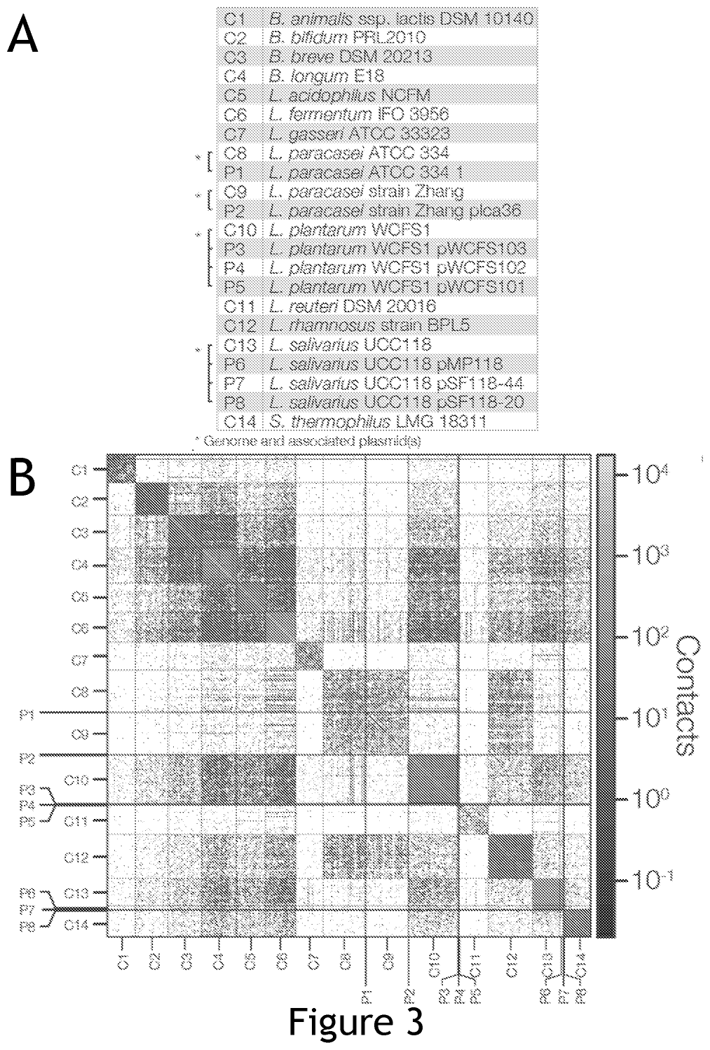

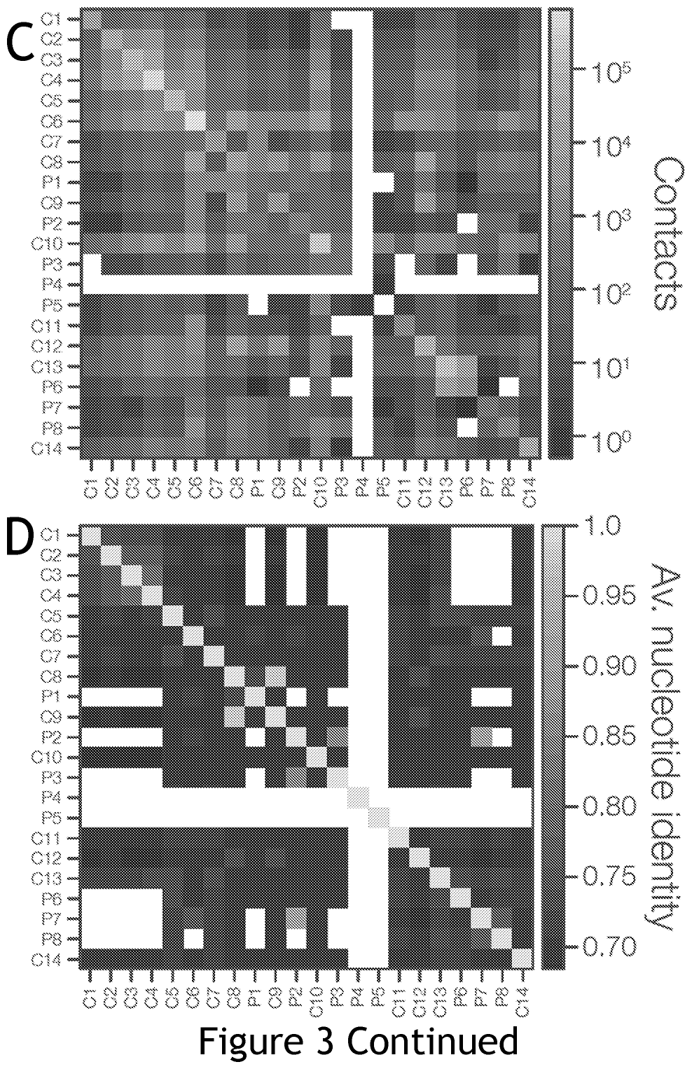

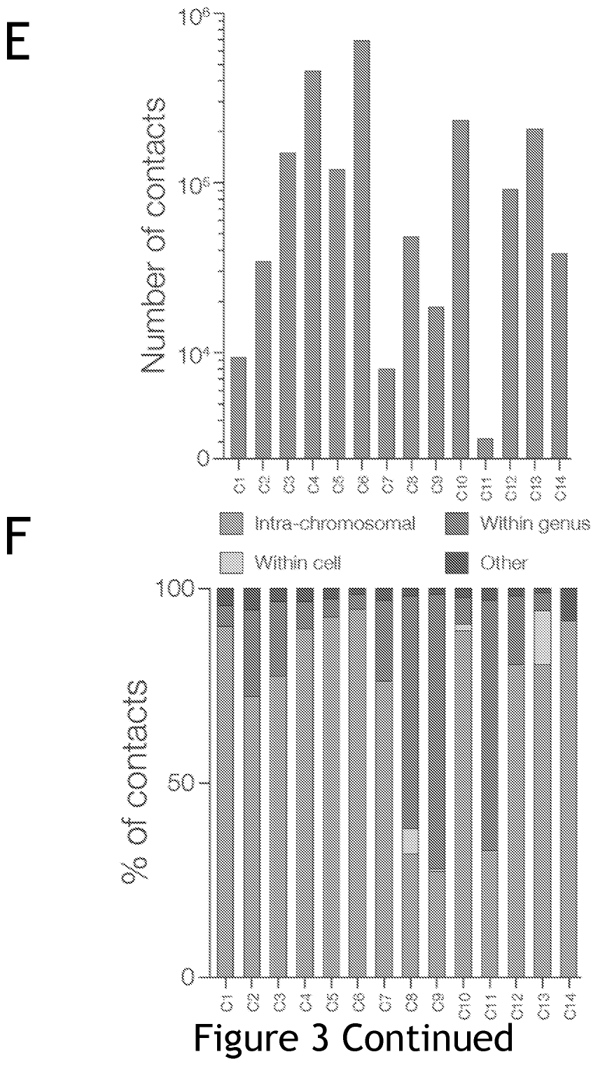

Results depicted by FIG. 3 show that the methods and workflows described above yield results demonstrating the identification of intra- and extra-chromosomal contacts in a probiotic sample. Genomic DNA from a probiotic food supplement sample, which contained 15 known bacterial strains (FIG. 3A), was applied to the methods and workflows described above and nanopore sequencing data was generated. Contact maps for the bacterial chromosomes and plasmids within the sample were prepared in accordance with the bioinformatics workflow above (FIGS. 3B and 3C). The plot of average nucleotide identity (FIG. 3D) reveals a low level of spurious interaction between species, most probably due to nanopore sequencing read mapping ambiguities. FIGS. 3E and 3F summarise the contacts for each bacterial chromosome. Plasmids were associated to the expected host genomes and intra-chromosomal interactions were identified, which were valuable for binning and hence assembly of the contact maps.

### Example 2

This Example describes a further exemplary laboratory workflow applicable to the present disclosure, a method for determining interactions between elements within a cell. In particular, methods are used to investigate interactions between genomic elements that are not adjacent in the primary sequence. In addition to simplifying assembly, when applied to metagenomic samples, the disclosed methods also provide a way to associate plasmids with their host genomes. Alternative protocols for determining interactions between elements within one or more DNA molecules use restriction digestion to fragment cross-linked DNA prior to performing proximity ligation. However, restriction digestion is time-consuming and also, the choice of restriction enzyme is influenced by the nucleotide composition of the genomes in the sample, which is not always known in advance—particularly when performing metagenomics investigations. The present disclosure provides a method that avoids restriction digestion by using mechanical fragmentation, for example bead-beating, to simultaneously lyse cells and fragment DNA. Bead beating may also be used to fragment DNA from lysed cells.

### Materials and Methods

### Sample Collection and Crosslinking

Approximately 2-3×10⁹ intact bacterial cells were collected and separated by methods known in the art. Cells were resuspended in a pre-mixed crosslinking buffer (1.2 mL PBS, 34 μL 37% formaldehyde (1% formaldehyde final concentration)) and incubated at room temperature for 30 minutes with occasional mixing. 170 μL of 1M glycine was added (125 mM final concentration of glycine) to quench the crosslinking reaction, and the sample was incubated for a further 20 minutes at room temperature. The sample was then centrifuged at 17000 g for 5 minutes, the supernatant was discarded and the crosslinked cell pellet (fixed cells) was washed with 1×TBS. The sample was centrifuged for a further 5 minutes at 17000 g and the supernatant was discarded. The pellet could then be stored at −80° C. for future use.

### Cell Lysis and End-Repair

The pellet of fixed cells should be thawed on ice if previously stored at −80° C. The cells (approximately 10 μL) were then resuspended in 200 μL beating solution as set out in Table 5. The total volume of the beating solution may be scaled up/down as required.

TABLE 5

| Reagent | Volume |
|---|---|
| 1 × TBS | 200 μL |
| 100 × Halt Protease Inhibitor | 4 μL |
| Triton X-100 | 2 μL |
| Total | 206 μL |

100 μL of 0.5 mm diameter glass beads (Qiagen) were then added to the resuspended cells and the sample was then vortexed for 3×5 minutes at the highest speed, each five minutes were separated by 2 minutes on ice. The sample was briefly centrifuged to collapse bubbles, and the sample was remixed by pipetting the sample solution up and down in order to produce a homogenous cell lysate. The lysate was then transferred to a new tube and centrifuged for five minutes at 17000 g. The supernatant was discarded and the pellet was resuspended in 500 μL 1×TBS. The sample was again centrifuged for 5 minutes at 17000 g and the supernatant was subsequently discarded. The pellet was resuspended in 50 μL H₂O, mixed thoroughly, and the DNA concentration was measured preferably by a Qubit (ThermoFisher Scientific). The end-repair reaction was formulated as set out in Table 6.

TABLE 6

| Reagent | Volume | |
|---|---|---|
| Up to 5 μg of chromatin DNA | x | μL |
| NEBNext End Repair Buffer (10×) | 10 | μL |
| NEBNext End Repair Enzyme mix | 5 | μL |
| H₂O | 85-x | μL |
| Total | 100 | μL |

The end-repair reaction was incubated at 20° C. for 30 minutes and then centrifuged for five minutes at 17000 g. The supernatant was discarded and the pellet was washed with 1×TBS. The sample was then briefly centrifuged at 17000 g and the supernatant was subsequently discarded. The pellet could then be stored at −20° C. for future use.

### Proximity Ligation

The pellet of end-repaired DNA was resuspended in 200 μL H₂O and quantified by Qubit. A T4 ligation was then set up as in Table 7 below.

TABLE 7

| Reagent | Volume | |
|---|---|---|
| H₂O | 1618-x | μL |
| 0.9 ug DNA in H2O | x | μL |
| 10 × T4 ligase buffer | 180 | μL |
| (Thermofisher) | | |
| Mix thoroughly | | |
| T4 DNA ligase | 6 | μL |
| (Thermofisher, | | |
| 30 weissU/uL) | | |
| Total | 1800 | μL |

The final concentration of DNA in the proximity ligation reaction is 0.5 ng/μL. The reaction was incubated at 22° C. for four hours with occasional mixing. The sample was then centrifuged at 17000 g for five minutes. 1375 μL of the supernatants was removed, leaving approximately 475 μL of supernatant remaining. 25 μL of 5 M NaCl was added to a final volume of 500 μL. Multiple 500 μL reactions may now be combined in the same sample tube if desired. The sample was then incubated overnight in order to decrosslink.

### Clean-Up

Per 500 μL decrosslinking reaction, the reagents set out in Table 8 were added in order.

TABLE 8

| Reagent | Volume | |
|---|---|---|
| 10% Tween-20 | 25 | μL |
| 10% Triton X-100 | 25 | μL |
| Lysozyme (250 mg/mL) | 5 | μL |
| RNaseA (Qiagen) | 2.5 | μL |
| Mix and incubate for 10 | | |
| minutes at room temperature | | |
| Qiagen 20 mg/mL | 12.5 | μL |
| Proteinase K | | |

The reaction was incubated for 30 minutes at 37° C. 170 μL of Qiagen buffer B2 as added to each reaction as above, and the reaction was then incubated at 50° C. for 30 minutes. DNA was then purified by phenol:chloroform:isoamyl-alcohol followed by isopropanol precipitation.

### Sequencing

The purified DNA can be either 1) directly prepared for sequencing using Oxford Nanopore Technologies' standard library preparation workflows/kits (this enables native DNA modification (epigenomics) to be retained in sequencing data generated); or, 2) amplified by PCR, using by Oxford Nanopore Technologies' PCR sequencing workflows/kits.

For nanopore-based sequencing application, the optimal PCR cycle DNA products were then size selected by gel-extraction or Bluepippin (SAGE Science) for PCR products larger than between 2 and 3 kb. Nanopore-based sequencing was then performed according to nanopore library preparation and sequencing protocols known in the art.

### Bioinformatics Workflow

A representative schematic of a bioinformatics analysis workflow that is applicable to the presently described exemplary method is provided by FIG. 2. In any of the described methods, the bioinformatics analysis workflow may be utilised for obtaining a metagenomics contact map from sequencing data derived using the methods applied to a metagenomics sample. In more detail, nanopore sequencing data is generated by the earlier described method to provide nanopore sequencing reads. The reads are first aligned to a collection of reference sequences for chromosomal and extra-chromosomal sequences, such as plasmids, using BWA-SW (Li H. and Durbin R. (2010) Fast and accurate long-read alignment with Burrows-Wheeler. Transform. Bioinformatics, Epub. [PMID: 20080505]). Each aligned read is filtered to retain the minimal collection of alignments that traverse the majority of the read. The reference genomes are then divided into equally sized bins and each aligned segment of a nanopore sequencing read is assigned a bin. Finally, the total number of bin-to-bin contacts is calculated from all nanopore sequencing reads and visualised in a contact map. Extra-chromosomal elements can be assigned to their host by determining which chromosome(s) share the most contacts with the element.

1. A method for detecting interactions between elements within one or more DNA molecules within a cell, wherein the elements are not adjacent in the primary DNA sequence, the method comprising:
a) providing a cell in which elements within one or more DNA molecules that are in close proximity are cross-linked;
b) simultaneously lysing the cell and mechanically fragmenting the DNA molecules within the cell;
c) proximity ligating the one or more fragmented DNA molecules;
d) reversing the crosslinks in the ligated DNA molecules;
e) sequencing the ligated DNA molecules; and
f) analysing the sequencing data to detect interactions between elements within the one or more DNA molecules within the cell.

2. A method according to claim 1, wherein the cells are lysed and the DNA molecules are fragmented by bead-beating.

3. A method for detecting interactions between elements within one or more DNA molecules within a cell or nucleus, wherein the elements are not adjacent in the primary DNA sequence, the method comprising:
a) providing a cell or nucleus in which elements within one or more DNA molecules that are in close proximity are cross-linked;
b) mechanically fragmenting the DNA molecules within the cell by bead beating;
c) proximity ligating the one or more fragmented DNA molecules;
d) reversing the crosslinks in the ligated DNA molecules;
e) sequencing the ligated DNA molecules; and
f) analysing the sequencing data to detect interactions between elements within the one or more DNA molecules within the cell.

4. A method according to claim 1, further comprising the initial step of cross-linking the DNA molecules and/or DNA-interacting proteins.

5. A method according to claim 4, wherein cross-linking is achieved by treating the cells with formaldehyde and/or disuccinimidyl glutarate (DSG).

6. A method according to claim 1, further comprising a step of blunt ending the DNA fragments produced in step (b) prior to step (c).

7. A method according to claim 1, further comprising a step of adding adaptors to the ends of the DNA fragments after step (c) or (d).

8. A method according to claim 7, wherein the adaptors are sequencing adaptors.

9. A method according to claim 8, wherein the sequencing adaptors are PCR sequencing adaptors.

10. A method according to claim 1, further comprising a step of purifying the DNA fragments after step (d).

11. A method according to claim 1, further comprising selecting DNA fragments of a desired size after step (b), (c) or (d).

12. A method according to claim 1, further comprising a step of enriching for one or more DNA molecules of interest prior to step (e).

13. A method according to claim 12, wherein DNA fragments of interest are enriched by hybridising one or more labelled oligonucleotides to one or more regions of interest within the DNA molecules, and selecting labelled DNA molecules.

14. A method according to claim 13, wherein the oligonucleotides are labelled with an affinity tag and labelled DNA molecules are selected by binding to the binding partner of the affinity tag.

15. A method according to claim 1, wherein sequencing is performed by a nanopore-based method.

16. A method according to claim 1, wherein the DNA molecules comprise chromosomal sequences and/or extra-chromosomal sequences.

17. A method according to claim 1, wherein the step of analysing the sequencing data comprises identifying concatenated sequences from different elements within the one or more DNA molecules thereby detecting interacting elements of one or more DNA elements.

18. A method according to claim 1, wherein the elements are loci within a chromosome.
